(19) United States
(12) Patent Application Publication
(10) Pub. No.: US 2003/0165582 A1
(43) Pub. Date: Sep. 4, 2003

(54) Preparations that contain an extract of the plant pistia stratiotes

(76) Inventors: Gilles Pauly, Nancy (FR); Louis Danoux, Saulxures Les Nancy (FR)

Correspondence Address: COGNIS CORPORATION, 2500 RENAISSANCE BLVD., SUITE 200, GULPH MILLS, PA, 19406

(21) Appl. No.: 10/297,728

(22) PCT Filed: Jun. 12, 2001
(86) PCT No.: PCT/EP01/06606

(30) Foreign Application Priority Data
Jun. 21, 2000 (FR) 00/07946

(51) Int. Cl. A61K 35/78

(52) U.S. Cl. 424/725

(57) ABSTRACT

Methods for treating substrates, including the skin and hair, by contacting the substrate with an extract of the Pistia Stratiotes plant are described, along with skin-care and hair-care compositions containing such extracts and processes for preparing such extracts.

### FIELD OF THE INVENTION

This invention relates generally to care preparations and more particularly to new skin-care and hair-care preparations and to their use in the cosmetics and pharmaceutical fields.

### PRIOR ART

Today, cosmetic preparations are available to the consumer in a variety of combinations. Consumers not only expect these cosmetics to have a certain care effect or to eliminate a certain deficiency, they are also increasingly demanding products which combine several properties and thus show an improved performance spectrum. There is a particular interest in substances which represent active principles that impart, for example, caring, anti-ageing and revitalizing properties to the skin and/or hair and, at the same time, positively influence, or at least do not adversely affect, the technical properties of the cosmetic product, such as storage stability, light stability and formulatability. In addition, consumers demand high dermatological compatibility and, above all, the use of natural products. In addition, it is desirable to obtain distinctly better products by combining already known active principles or by discovering new applications for already known classes of substances. However, one disadvantage in this regard is often that a combination of active principles is only obtained when different plant extracts are simultaneously used in different quantity ratios.

Extracts of plants and their ingredients are being increasingly used in cosmetic and pharmaceutical products. For many years, plant extracts have been used for medicinal purposes and also for cosmetic purposes in many different cultures. These plant extracts were often known only for very specific individual effects which limited their scope of application.

### DESCRIPTION OF THE INVENTION

The problem addressed by the present invention was to provide plant extracts from a plant which could be used in cosmetic or even pharmaceutical products and which, besides care properties, would show above all a preventive and healing effect against signs of ageing of the skin, would have a reactivating and revitalizing effect and, at the same time, could be used as anti-cellulitis slimming aids.

Another problem addressed by the present invention was to provide preparations which would contain active ingredients from renewable raw materials and, at the same time, could be widely used as care preparations both in skin care and in hair care.

The present invention relates to preparations extracted from the plant Pistia stratiotes. It has surprisingly been found that the use of extracts of Pistia stratiotes leads to products which combine good skin- and hair-care and -protecting properties with high dermatological compatibility. The preparations thus obtained are distinguished by particularly good skin-care effects. Besides slimming properties and an anti-cellulitis effect, they also show a preventive and healing effect against signs of skin ageing and revitalizing and reactivating activity on the skin and hair.

These multiple applications of the preparations according to the invention from the renewable raw material of the plant Pistia stratiotes make it very attractive both to the market and to the consumer. Accordingly, the complex problem addressed by the invention has been solved by the use of an extract of the plant Pistia stratiotes.

Plants in the context of the present invention are understood to be both whole plants and parts thereof (leaves, blossoms, roots) and mixtures thereof.

Pistia stratiotes

The extracts to be used in accordance with the invention are obtained from plants of the Araceae family or the aroids family. More specifically, they are extracts of the plant Pistia stratiotes. This plant is a broad-leaved, floating rosette plant with roots which is also known as water lettuce. It is widespread in the tropics and subtropics. At its lower end, the spadix—surrounded by an ascidiform sheath—carries a female flower and, at its upper end, separated by a kind of frill, a male flower which consists of two stamens joined to form a synandrium. The inflorescences are so inconspicuous that they are almost hidden by the broad oval leaves (cf. also Herder, Lexikon der Biologie, Vol. 6, Spektrum Akademischer Verlag, Heidelberg, Berlin, Oxford).

In traditional medicine, this plant has already been used for treating dysuria and as a diuretic. The roots were used against inflammations and burns. In the form of a mixture with rice and coconut oil, it was administered in cases of dysentery. In conjunction with rose water and sugar, it was used to relieve asthma and coughs. In Chinese folk medicine, the whole plant was used for some time against ulcers and eczema and against syphilis.

Japanese patent JP 10139639 describes a hair growth inhibitor containing a combination of many different plant extracts including an extract of the plant Pistia stratiotes. This is no indication as to which of the combined plant extracts or which combination of the plant extracts present is responsible for this effect.

Extraction

The extracts to be used in accordance with the invention may be prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Fresh or dried plants or parts thereof are suitable as the starting material although plants and/or plant parts which may be mechanically size-reduced before extraction are normally used. Any size reduction methods known to the expert, for example comminution with a bladed tool, may be used.

Preferred solvents for the extraction process are organic solvents, water or mixtures of organic solvents and water, more particularly low molecular weight alcohols, esters, ethers, ketones or halogenated hydrocarbons with more or less large water contents (distilled or non-distilled), preferably aqueous alcoholic solutions with more or less large water contents. Extraction with water, methanol, ethanol, propanol, butanol and isomers thereof, acetone, propylene glycols, polyethylene glycols, ethyl acetate, dichloromethane, trichloromethane and mixtures thereof is particularly preferred. The extraction process is generally carried out at 20 to 100° C. and preferably at 80 to 100° C., more particularly at the boiling temperature of the solvents or solvent mixtures. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried plants or dried plant parts (optionally degreased) are in the range from 1 to 20, preferably 4 to 16 and more particularly 8 to 12% by weight. The present invention includes the observation that the extraction conditions and the yields of the final extracts may be selected according to the desired application. If desired, the extracts may then be subjected, for example, to spray drying or freeze drying.

The quantity of plant extracts used in the preparations mentioned is governed by the concentration of the individual ingredients and by the way in which the extracts are used. In general, the total quantity of plant extract present in the preparations according to the invention is 0.01 to 25% by weight, preferably 0.03 to 5% by weight and more particularly 0.03 to 0.6% by weight, based on the final preparation, with the proviso that the quantities add up to 100% by weight with water and optionally other auxiliaries and additives. The total content of auxiliaries and additives may be 1 to 50% by weight and is preferably 5 to 40% by weight, based on the final cosmetic and/or pharmaceutical preparation. The preparations may be produced by standard cold or hot processes but are preferably produced by the phase inversion temperature method.

Active substance in the context of the invention is based on the percentage content of substances and auxiliaries and additives present in the preparation except for the water additionally introduced.

Extracts

The extracts of the plant Pistia stratiotes according to the invention generally contain ingredients from the group consisting of sterols, carotinoids, proteins, carbohydrates, fats, vitamins and/or mineral salts. The extracts vary in their composition according to the starting material and extraction method selected.

Sterols in the context of the invention are steroids which can be isolated from the plant Pistia stratiotes. More particularly, they are steroids which only bear a hydroxy group at C-3, i.e. formally are alcohols. In addition, the sterols containing 27 to 30 carbon atoms generally have a C═C double bond in the 5/6 position and occasionally even/or in the 7/8, 8/9 and other positions (for example 22/23). Particularly preferred sterols isolated from the plant Pistia stratiotes are such sterols as stigmasterol, stigmastearyl stearates and/or stigmast-4,22-dien-3-one.

Carotinoids in the context of the invention are those which can be isolated from the plant Pistia stratiotes. More particularly, they are substances which, chemically, represent 11× to 12× unsaturated tetraterpenes with a basic skeleton containing 9 conjugated double bonds, 8 methyl branches (including the possible ring structures) and a β-ionone structure at one end of the molecule, but which differ in structure at the other end of the molecule. Typical carotinoids are, for example, β-carotene or provitamin A, α-carotene, lutein, cryptoxanthine, zeaxanthine and lycopene. The percentage content of carotinoids in the extract of the plant Pistia stratiotes is between 300 and 400 mg/kg extract dry weight and more particularly between 320 and 360 mg per kg dry weight.

Proteins in the context of the invention are those which can be isolated from the plant Pistia stratiotes. Their percentage content based on extract dry weight is between 15 and 25 mg/kg and more particularly between 20 and 22 mg/g. Proteins are a constituent of the plant plasma and accordingly can be found in all plant parts.

Carbohydrates in the context of the invention are those which can be isolated from the plant Pistia stratiotes. Preferred carbohydrates are cellulose, glucan, inulin, agar agar, carrageen and alginic acid.

Fats in the context of the invention are those which can be isolated from the plant Pistia stratiotes. They are solid, semisolid or liquid, more or less viscous triglycerides of the plant which, chemically, consist essentially of mixed glycerol esters of higher fatty acids with an even number of carbon atoms. A preferred fatty acid is palmitic acid. The percentage content of fats in the extract dry matter is between 6 and 10 mg/kg and preferably between 8 and 9 mg/kg.

Vitamins in the context of the invention are those which can be isolated from the plant Pistia stratiotes. More particularly, these vitamins—besides retinol and dehydroretinol (vitamins A1 and A2)—are preferably ascorbic acid (vitamin C), α-tocopherol (vitamin E), thiamin (vitamin B1), riboflavin (vitamin B2), pyridoxal (vitamin B6), folic acid (vitamin B9), niacin (vitamin B3) and pantothenate which are isolated in varying amounts.

In the context of the present invention, the extracts of the plant Pistia stratiotes contain minerals in the form of salts of the alkali or alkaline earth metals. The predominant metals are sodium, potassium or calcium. The alkali or alkaline earth metals occur in the form of their salts, but predominantly in the form of their halides, oxides or hydroxides, phosphates, carbonates, sulfates or nitrates.

The present invention also relates to the use of the extracts of the plant Pistia stratiotes in skin care and/or hair care preparations.

Care Preparations

Care preparations in the context of the invention are understood to be skin care and hair care preparations. These preparations include inter alia a cleaning effect and restorative effect on the skin and hair.

In addition, the preparations according to the invention combine an excellent skin care effect with high dermatological compatibility. They also show high stability, particularly against oxidative decomposition of the products.

The preparations according to the invention may be applied topically and orally in the form of tablets, dragées, capsules, juices, solutions and granules.

The present invention also relates to the use of extracts of Pistia stratiotes in slimming aids for the skin with anti-cellulitis activity

Cellulitis—which is also known as orange skin—forms in the subcutis. This layer of skin represents a loose connective tissue with incorporations of fatty cell groups (fatty lobules). Commensurate with its structure, the subcutis is a displacement and connecting layer between skin and substrate, a nutrient and water store, the location of the pressure receptors, the site of the fat and carbohydrate metabolism and the region where relatively large vessels pass through to the skin surface. By virtue of their high lipolytic activity, the extracts according to the invention are used in slimming and anti-cellulitis preparations. The lipolytic activity is a measure of the body's own degradation of fat in the adipocytes.

The present invention also relates to the use of extracts of Pistia stratiotes in care preparations for the preventive or healing treatment of signs of skin ageing. Another name for care preparations of this type is anti-ageing preparations. Such signs of ageing include, for example, any type of wrinkling or lining. The treatments include slowing down of the skin ageing processes. The ageing signs can have various causes. More particularly, they are caused by UV-induced skin damage. In one particular embodiment of the invention, the care preparations are used for the treatment of UV-induced ageing of the skin. In another particular embodiment, the care preparations according to the invention are used for the treatment of induced apoptosis and correspondingly induced signs of skin ageing attributable to a lack of growth factors.

In the context of the invention, apoptosis is understood to be the controlled cell death of certain unwanted or damaged cells. It is an active cell process (suicide on command). Apoptosis is initiated by oxidative stress (UV radiation, inflammation), by a deficiency of growth factors or by toxins (pollutants, genotoxins, etc.). In the skin ageing process, for example, apoptosis of the skin cells can be induced by a deficiency of growth factors in the skin. In the apoptosis-affected cells, the nuclear DNA is degraded by the specific enzyme endonuclease and the DNA fragments are channeled into the cytoplasm. In principle, growth factors are understood to be genetic or extrinsic growth factors which stimulate the growth of skin and hair cells. They include, for example, hormones and chemical mediators or signal molecules. Examples are polypeptide growth factors and glycoprotein growth factors. Mention is made here of the epidermal growth factor (EGF), which consists of 53 amino acids and hence represents a polypeptide growth factor, or fibrillin which is a glycoprotein. Other growth factors are, for example, urogastrone, laminin, follistatin and heregelin.

The present invention also relates to the use of extracts of Pistia stratiotes in protective and restorative skin care and hair care preparations with revitalizing and reactivating activity. This way of using the care preparations has a positive effect, for example, on the adverse effects of environmental contamination of the skin and/or hair by reactivating the natural functions of the skin and/or hair and by making the skin and/or hair more resistant. The revitalizing and reactivating activity of extracts of the plant Pistia stratiotes counteracts apoptosis. In principle, the extracts according to the invention may be used as protective and restorative care preparations for any preparations which are used to prevent damage or to treat damage to the skin and/or hair and hence in skin and hair care. Another use in this field is application to sensitive skin damaged by allergies or other factors. The skin damage can have various causes.

The present invention also relates to a process for the preparation of an extract of the plant Pistia stratiotes in which solvents or mixtures of solvents selected from the group consisting of distilled or non-distilled water, low molecular weight alcohols, esters, ethers, hydrocarbons, ketones or halogenated hydrocarbons are used as the extraction medium for extraction. According to the invention, particularly preferred extractants are distilled or non-distilled water, low molecular weight alcohols, such as methanol, ethanol, propanol, butanol and isomers thereof, as pure solvents or as solvents of technical purity and as aqueous solutions, in which case the water content may vary according to the alcohol and the extraction method. Extraction is generally carried out at 20 to 100° C., preferably at 80 to 100° C. and more particularly at the boiling temperature of the solvent or solvent mixture.

The preparations according to the invention may be used for the production of cosmetic and/or pharmaceutical preparations such as, for example, hair shampoos, hair lotions, foam baths, shower baths, creams, gels, lotions, alcohol and water/alcohol solutions, emulsions, wax/fat compounds, stick preparations, powders or ointments. In addition, the preparations according to the invention may also be incorporated in tablets, dragées, capsules, juices, solutions and granules for oral application. Oral application is particularly preferred in care preparations with slimming and anti-cellulitis properties for the skin. These preparations may additionally contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, preservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

products of the addition of 2 to 30 moles of ethylene oxide and/or 0 to 5 moles of propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alkyl group and ethoxylated analogs thereof;

addition products of 1 to 15 moles of ethylene oxide onto castor oil and/or hydrogenated castor oil;

addition products of 15 to 60 moles of ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 moles of ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 moles of ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as re-fatting agents for cosmetic formulations from DE 20 24 051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 moles of ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 moles of ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethylimidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids; N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxy-substituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinylimidazole polymers such as, for example, Luviquat® (BASF), condensation products of polyglycols and amines, quaternized collagen polypeptides such as, for example, Lauryidimonium Hydroxypropyl Hydrolyzed Collagen (Lamequat® L, Grunau), quaternized wheat poly-peptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacryl-amide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil. 108, 95 (1993).

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

Biogenic agents in the context of the invention are additionally those which do not originate from the plant Pistia stratiotes such as, for example, tocopherol acetate, tocopherol palmitate, ascorbic acid, deoxyribonucleic acid and fragmentation products thereof, retinol, bisabolol, allantoin, phytantriol, panthenol, AHA acids, amino acids, ceramides, pseudoceramides, essential oils, plant extracts and additional vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers. Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolau rate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Färbemittel” of the Farbstoffkommission der Deutschen Forschungsgemeinschaft, Verlag Chemie, Weinheim, 1984, pages 81 to 106. These dyes are normally used in concentrations of 0.001 to 0.1% by weight, based on the mixture as a whole.

### EXAMPLES

### Example 1

### Extraction of the Plants with Distilled Water

300 g of dried Pistia stratiotes plants were coarsely size-reduced in a bladed size reducing machine and transferred to a glass reactor containing 4.5 liters of distilled water. The infusion was heated to between 80 and 85° C. and extracted with stirring for 1 h at that temperature. The mixture was then cooled to 20° C. and centrifuged for 15 mins. at a speed of 5000 G. The supernatant colloidal liquid was separated from the residue by filtration through depth filters with a mean porosity of 450 nm (from Seitz of Bordeaux, France). The extract was spray-dried at a starting temperature of 185° C. and an end temperature of 80° C. The yield of dry product was 8.8 to 12.1% by weight, based on the dry weight of plants used.

### Example 2

### Extraction of the Plants with Aqueous Methanol

Example 1 was repeated except that extraction was carried out with 3 liters of 80% by weight aqueous methanol. Extraction was carried out with stirring under reflux for 1 hour at boiling temperature and the extract was further processed as described above. Filtration was carried out as described in Example 1. Thereafter the alcohol was removed under reduced pressure at 45° C. and the green-brown residue was then spray-dried as described. The yield of dry product was 7 to 8% by weight, based on the dry weight of plants used.

### Example 3

### Extraction of the Plants with Aqueous Ethanol

Example 1 was repeated except that extraction was carried out with 3 liters of 96% by weight aqueous ethanol. Extraction was carried out with stirring under reflux for 1 hour at boiling temperature and the extract was further processed as described above. Filtration was carried out as described in Example 1 and the residue was re-washed with 0.75 liter of 96% by weight aqueous ethanol. Thereafter the alcohol was removed under reduced pressure at 45° C. and the green residue was then dried at 50° C. The yield of dry product was 1.5 to 5.3% by weight, based on the dry weight of plants used.

### Example 4

### Inhibition of Aptosis Induction

Background: In contrast to necrosis, apoptosis is understood to be the natural, controlled cell death of certain unwanted or damaged cells. It is an active cell process (suicide on command). Apoptosis is initiated by oxidative stress (UV radiation, inflammation), by a deficiency of growth factors or by toxins (pollutants, genotoxins, etc.). In the skin ageing process, for example, apoptosis of the skin cells can be induced by a deficiency of growth factors in the skin. In the apoptosis-affected cells, the nuclear DNA is degraded by the specific enzyme endonuclease and the DNA fragments are channeled into the cytoplasm.

Method: The ability of the plant extracts of Pistia stratiotes to prevent apoptosis induced by a deficiency of growth factors in human skin cells was investigated. The test was conducted in vitro on human fibroblasts and human keratinocytes. The human cells were cultivated in a nutrient medium (DMEM=Dulbecco Minimum Essential Medium from Life Technologie S.a.r.l.) containing 10% of foetal calf serum (from Dutcher). Bromodeoxyuridine (BrdU) was added to this nutrient medium. It was incorporated in the DNA and was subsequently used to detect the DNA fragments in the cytoplasm. After two days' incubation, the nutrient medium was replaced by nutrient medium (DMEM) with no foetal calf serum. The active substance to be tested was added. For the plant extract, two different batches, i.e. two different extracts (batches A and B) obtained by the same extraction method, were tested. For comparison, a cell sample was incubated without any active substance to be tested (quantities and concentrations shown in Tables 1 and 2).

After incubation for one or two days at 37° C., the cells were recovered by trypsination using the Dunnebacke and Zitcer method described in Cell and Tissue Culture, Ed.: J. Paul, Churchill Livingstone, 1975, p. 226. After the trypsin treatment, the cells were centrifuged and counted. The BrdU content in DNA fragments from the cytoplasm was then determined by the ELISA Test (ELISA Kit from Roche). The BrdU content is a measure of the DNA fragments chanelled into the cytoplasm from the nucleus, the cell core. The results were based on one million cells and were expressed in percent by comparison with the control.

TABLE 1
Number of cells and content of DNA fragments in the cytoplasm after treatment of
human fibroblasts with Pistia stratiotes extracts

| | |
|---|---|
| Batch | Batch |

| Cell counting | A | B | Content of DNA fragments | A | B |
|---|---|---|---|---|---|
| Control | 100 | 100 | Control | 100 | 100 |
| Extract of Ex. 1; 0.01% by | 101 | 97 | Extract of Ex. 1; 0.01% by | 72 | 66 |
| weight | | | weight | | |
| Extract of Ex. 1; 0.03% by | 97 | 101 | Extract of Ex. 1; 0.03% by | 68 | 50 |
| weight | | | weight | | |
| Control | 100 | 100 | Control | 100 | 100 |
| Extract of Ex. 3; 0.01% by | 104 | 106 | Extract of Ex. 3; 0.01% by | 74 | 63 |
| weight | | | weight | | |
| Extract of Ex. 3; 0.03% by | 124 | 112 | Extract of Ex. 3; 0.03% by | 46 | 50 |
| weight | | | weight | | |

TABLE 2
Number of cells and content of DNA fragments in the cytoplasm after treatment of
human keratinocytes with Pistia stratiotes extracts

| | |
|---|---|
| Batch | Batch |

| Cell counting | A | B | Content of DNA fragments | A | B |
|---|---|---|---|---|---|
| Control | 100 | 100 | Control | 100 | 100 |
| Extract of Ex. 1; 0.01% by | 92 | 92 | Extract of Ex. 1; 0.01% by | 58 | 64 |
| weight | | | weight | | |
| Extract of Ex. 1; 0.03% by | 89 | 92 | Extract of Ex. 1; 0.03% by | 44 | 61 |
| weight | | | weight | | |
| Control | 100 | 100 | Control | 100 | 100 |
| Extract of Ex. 3; 0.01% by | 94 | 109 | Extract of Ex. 3; 0.01% by | 87 | 64 |
| weight | | | weight | | |
| Extract of Ex. 3; 0.03% by | 93 | 111 | Extract of Ex. 3; 0.03% by | 73 | 60 |
| weight | | | weight | | |

Results: It can be seen from the results set out in Tables 1 and 2 that the use of extracts from the plant Pistia stratiotes reduces apoptosis in human cell cultures in vitro. The content of free DNA fragments in the cytoplasm and hence the degree of destroyed DNA in the cell nucleus and the degree of apoptosis decrease with increasing concentration of Pistia stratiotes extract. The cell counts document the fact that the plant extracts according to the invention are non-toxic and do not lead to cell death. The number of intact cells present has hardly changed whereas the content of DNA fragments in the cytoplasm is reduced by comparison with the control under the influence of the plant extract. Selective cell death is suppressed by these plant extracts. The plant extracts have an anti-ageing effect on human skin cells.

### Example 5

### Lipolytic Activity on Human Adipocytes

Background: Lipolysis is the term for the body's own degradation of fats present as reserves in the adipocytes (fatty cells). Adipocytes are enzymatically split by lipases into smaller molecular fragments, the fatty acids and glycerol. The free fatty acids are then used by the muscle cells for gaining energy.

Method: The adipocytes were isolated from human subcutaneous tissue by the standard Rodbell method (J. of Biolog. Chem.; 1964, 239, 375-380). The extract of Examples 1 to 3 each with two extracts (batches A and B) and the comparison substances were dissolved in a Hanks reference medium with a particular salt concentration (Cell and Tissue Culture, Ed: J. Paul, Churchill and Livingstone, 1975, page 484) and then contacted with the isolated adipocytes for 90 minutes at 37° C. Two to six adipocyte preparations were investigated. The percentage increase in glycerol released was spectrophotometrically determined in the supernatant of the medium by the Carpéné method (J. de Pharmacologie; 1981; 12;219-224). The same medium without any substance to be tested was administered as reference (reference value=0).

TABLE 3
Lipolytic activity on human adipocytes

| | | No. of pre- | Concentration | % Increase in |
|---|---|---|---|---|
| Substance | Batch | parations | % by weight | glycerol released |
| Extract of | A | 6 | 0.20 | 49 |
| Example 1 | | 6 | 0.50 | 125 |
| | B | 6 | 0.20 | 36 |
| | | 6 | 0.50 | 108 |
| Extract of | A | 4 | 0.02 | 35 |
| Example 2 | | 4 | 0.05 | 23 |
| | | 4 | 0.10 | 51 |
| | B | 4 | 0.02 | 21 |
| | | 4 | 0.05 | 41 |
| | | 4 | 0.10 | 97 |
| Extract of | A | 4 | 0.02 | 11 |
| Example 3 | | 4 | 0.05 | 31 |
| | | 4 | 0.10 | 171 |
| | | 2 | 0.20 | 82 |
| | | 2 | 0.50 | 212 |
| | B | 4 | 0.10 | 13 |
| | | 4 | 0.20 | 32 |
| | | 4 | 0.60 | 95 |
| Theophylline | | 14 | | 187 |
| (1.0 mM) | | | | |
| Isoprenalin | | 14 | | 129 |
| (0.1 μm) | | | | |

The results of the tests show that the Pistia stratiotes extracts of Examples 1 to 3 show increasing, concentration-dependent lipolytic activity on human adipocytes in vitro because the percentage increase in the glycerol released increases with increasing plant extract concentration. These results reflect the effect of the substances as slimming aids with anti-cellulitis activity.

### Example 6

### Skin Regenerating and Revitalizing Activity

The object of this test is to demonstrate the regenerating and revitalizing activity of extracts of Pistia stratiotes on human fibroblast cultures in vitro.

Method 1: Effects on Cell Growth

Human fibroblasts were inoculated with 10% by weight of foetal calf serum in a defined nutrient medium (DMEM=Dulbecco Minimum Essential Medium, a product of Life Technologie S.a.r.l.) and incubated for 24 h at 37° C. in a 5% CO₂ atmosphere. The nutrient medium containing foetal calf serum was then replaced by a nutrient medium of DMEM without foetal calf serum. Active substance in the form of the Pistia stratiotes extracts of Examples 1 to 3 was then added to this nutrient medium in various concentrations. For comparison, a test series of human fibroblasts with no active substance was incubated as control. After the fibroblasts had been incubated for three days in the nutrient medium, growth and metabolic activity were evaluated by counting the cells with a particle counter and determining the intracellular content of ATP by Vasseur's method (Journal Français Hydrologie, 1981, 9, 149-156). With concentrations of 0.003 to 0.01% by weight of plant extract of Examples 1 to 3, an increase in the percentage ATP content of between 16% and 43% was obtained by comparison with the control.

The study shows that the Pistia stratiotes extracts of Examples 1 to 3 stimulate the growth and metabolism of the human fibroblasts in vitro to a considerable extent.

Method 2: Improvement of Viability

The test was carried out on human fibroblasts. It enables a certain number of parameters to be quantitatively determined on the resting cells. The cultivation of the cells corresponds to the cultivation of method 1 except for the incubation time. The incubation time for this test was 72 h. Viability was evaluated by colorimetric determination of the percentage protein content by Bradford's method (Anal. Biochem. 1976, 72, 248-254), by determination of the percentage glutathione content (GSH) with a fluorescent probe, orthophthaldehyde, by Hissin and Hilf's method (Anal. Biochem. 1976, 74, 214-216). The glutathione is produced by cells in order to be able to react directly against oxidative stress and environmental influences, such as high heavy metal levels. Accordingly, an increased percentage content of reduced glutathione after treatment of the cells with the extracts of Examples 1 to 3 is a measure of the increased viability of the cells under the effect of external stress and other challenges. The test was carried out three times and then repeated twice so that there were six results per plant extract and hence per batch which were all averaged. The results were expressed in percent by comparison with the control.

TABLE 4
Increase in the protein content in human fibroblasts after incubation
with extracts of Examples 1 to 3 by comparison with incubation
without plant extract

| | | Concentration | Protein content |
|---|---|---|---|
| Substance | Batch | % by weight | in % |
| Control | | | 100 |
| Extract of Example 1 | A | 0.01 | 113 |
| | | 0.03 | 121 |
| | B | 0.01 | 139 |
| | | 0.03 | 143 |
| Extract of Example 2 | A | 0.01 | 119 |
| | | 0.03 | 133 |
| | B | 0.01 | 128 |
| | | 0.03 | 140 |
| Extract of Example 3 | A | 0.01 | 124 |
| | | 0.03 | 125 |
| | B | 0.01 | 128 |
| | | 0.03 | 149 |

The extracts of Pistia stratiotes of Examples 1 to 3 in concentrations of 0.01 to 0.03% by weight increase the content of proteins in human fibroblasts in comparison with the control by 21 to 49% in in vitro tests. The average glutathione increase was 30%. These results show that the extracts of Pistia stratiotes have a high capacity for stimulating the metabolism of fibroblasts. The extracts show regenerating and revitalizing activity of human fibroblasts and, accordingly, may be used as energy sources and as anti-ageing agents in cosmetic and pharmaceutical preparations.

The effects and positive activities of the Pistia stratiotes extracts therefore contain a very strong

activating (stimulating), revitalizing and regenerating activity on the metabolism,

apoptosis-inhibiting activity and hence anti-ageing activity and

a strong lipolytic activity which justifies their use in slimming care preparations.

### Example 7

### Exemplary Formulations of Cosmetic Products Containing Pistia stratiotes Extracts

The Pistia stratiotes extracts obtained in accordance with Examples 1 to 3 were used in the following formulations according to the invention K1 to K21 and 1 to 25. The cosmetic preparations thus produced showed very good skin-care properties coupled with high dermatological compatibility in relation to the comparison formulations C1, C2 and C3. In addition, the preparations according to the invention proved to be stable to oxidative decomposition.

TABLE 5
Soft cream formulations K1 to K7
(All quantities in % by weight, based on the cosmetic preparation)

| INCI name | K1 | K2 | K3 | K4 | K5 | K6 | K7 | C1 |
|---|---|---|---|---|---|---|---|---|
| Glyceryl Stearate (and) Ceteareth-12/20 (and) | 8.0 | 8.0 | 8.0 | 8.0 | 8.0 | 8.0 | 8.0 | 8.0 |
| Cetearyl Alcohol (and) Cetyl Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 3.0 | 3.0 | 3.0 | 3.0 | 3.0 | 3.0 | 3.0 | 3.0 |
| Glycerin (86% by weight) | 3.0 | 3.0 | 3.0 | 3.0 | 3.0 | 3.0 | 3.0 | 3.0 |
| Extracts of Examples 1-3 | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 6
Night cream formulations K8 to K14
(All quantities in % by weight, based on the cosmetic preparation)

| INCI name | K8 | K9 | K10 | K11 | K12 | K13 | K14 | C2 |
|---|---|---|---|---|---|---|---|---|
| Polyglyceryl-2 Dipolyhydroxystearate | 4.0 | 4.0 | 4.0 | 4.0 | 4.0 | 4.0 | 4.0 | 5.0 |
| Polyglyceryl-3 Diisostearate | 2.0 | 2.0 | 2.0 | 2.0 | 2.0 | 2.0 | 2.0 | 2.0 |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 8.0 | 8.0 | 8.0 | 8.0 | 8.0 | 8.0 | 8.0 | 8.0 |
| Dicaprylyl Ether | 5.0 | 5.0 | 5.0 | 5.0 | 5.0 | 5.0 | 5.0 | 5.0 |
| Magnesium sulfate | 1.0 | 1.0 | 1.0 | 1.0 | 1.0 | 1.0 | 1.0 | 1.0 |
| Glycerin (86% by weight) | 5.0 | 5.0 | 5.0 | 5.0 | 5.0 | 5.0 | 5.0 | 5.0 |
| Extract of Example 1-3 | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 7
W/O body lotion formulations K15 to K21.
(All quantities in % by weight, based on the cosmetic preparation)

| INCI name | K15 | K16 | K17 | K18 | K19 | K20 | K21 | C3 |
|---|---|---|---|---|---|---|---|---|
| PEG-7 Hydrogenated Castor Oil | 7.0 | 7.0 | 7.0 | 7.0 | 7.0 | 7.0 | 7.0 | 7.0 |
| Decyl Oleate | 7.0 | 7.0 | 7.0 | 7.0 | 7.0 | 7.0 | 7.0 | 7.0 |
| Cetearyl Isononanoate | 7.0 | 7.0 | 7.0 | 7.0 | 7.0 | 7.0 | 7.0 | 7.0 |
| Glycerin (86% by weight) | 5.0 | 5.0 | 5.0 | 5.0 | 5.0 | 5.0 | 5.0 | 5.0 |
| MgSO₄.7H₂O | 1.0 | 1.0 | 1.0 | 1.0 | 1.0 | 1.0 | 1.0 | 1.0 |
| Extract of Example 1-3 | 1.5 | 1.5 | 1.5 | 1.5 | 1.5 | 1.5 | 1.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

¹⁾Deoxyribonucleic acid: molecular weight ca. 70,000, purity (determined by spectrophotomeric measurement of absorption at 260 nm and 280 nm): at least 1.7

TABLE 8
Formulations
Cosmetic preparations conditioner (all quantities in % by weight, based on the
cosmetic preparation, water, preservative add up to 100% by weight)

| Composition (INCI) | 1 | 2 | 3 | 4 | 5 | 6 |
|---|---|---|---|---|---|---|
| Dehyquart ® A | 4.0 | 4.0 | | | 3.0 | |
| Cetrimonium Chloride | | | | | | |
| Dehyquart L ® 80 | | | 1.2 | 1.2 | | 1.0 |
| Dococoylmethylethoxymonium Methosulfate (and) Propyleneglycol | | | | | | |
| Eumulgin ® B2 | 0.8 | | — | 0.8 | — | 1.0 |
| Ceteareth-20 | | | | | | |
| Eumulgin ® VL 75 | — | 2.0 | 2.0 | — | 0.8 | — |
| Lauryl Glucoside (and) Polyglyceryl-2 Polyhydroxystearate (and) Glycerin | | | | | | |
| Lanette ® O | 3.0 | 3.0 | 3.0 | 3.0 | 3.0 | 3.0 |
| Cetearyl Alcohol | | | | | | |
| Cutina ® GMS | — | 0.5 | — | 0.5 | — | 1.0 |
| Glyceryl Stearate | | | | | | |
| Lamesoft ® PO 65 | | — | 3.0 | — | — | 3.0 |
| Coco-Glucoside (and) Gyceryl Oleate | | | | | | |
| Cetiol ® J 600 | — | 0.5 | — | 1.0 | — | 1.0 |
| Oleyl Eructae | | | | | | |
| Eutanol ® G | — | — | 1.0 | — | — | 1.0 |
| Octyldodecanol | | | | | | |
| Nutrilan ® Keratin W | 5.0 | — | — | 2.0 | — | — |
| Hydrolyzed Keratin | | | | | | |
| Generol ® 122 N | — | — | — | — | 1.0 | 1.0 |
| Soya Sterol | | | | | | |
| Pistia stratiotes extract | 1.0 | 1.0 | 1.0 | 1.0 | 1.0 | 1.0 |
| Copherol ® 12250 | — | — | 0.1 | 0.1 | — | — |
| Tocopheryl Acetate | | | | | | |

(1-4) Hair rinse, (5-6) hair treatment

Formulations for conditioners II.
Cosmetic preparations conditioner (all quantities in % by weight, based on the
cosmetic preparation, water, preservative add up to 100% by weight)

| Composition (INCI) | 7 | 8 | 9 | 10 |
|---|---|---|---|---|
| Texapon ® NSO | 38.0 | 38.0 | 25.0 | — |
| Sodium Laureth Sulfate | | | | |
| Texapon ® SB 3 | — | — | 10.0 | — |
| Disodium Laureth Sulfosuccinate | | | | |
| Plantacare ® 818 | 7.0 | 7.0 | 6.0 | — |
| Coco Glucosides | | | | |
| Plantacare ® PS 10 | — | — | — | 20.0 |
| Sodium Laureth Sulfate (and) Coco Glucosides | | | | |
| Dehyton ® PK 45 | — | — | 10.0 | — |
| Cocamidopropyl Betaine | | | | |
| Lamesoft ® PO 65 | 3.0 | | | 4.0 |
| Coco-Glucosiode (and) Glyceryl Oleate | | | | |
| Lamesoft ® LMG | — | 5.0 | — | — |
| Glyceryl Laurate (and) Potassium Cocoyl Hydrolyzed Collagen | | | | |
| Euperlan ® PK 3000 AM | — | 3.0 | 5.0 | 5.0 |
| Glycol Distearate (and) Laureth-4 (and) Cocamidopropyl Betaine | | | | |
| Pistia stratories extract | 1.0 | 1.0 | 1.0 | 1.0 |
| Arlypon ® F | 3.0 | 3.0 | 1.0 | — |
| Laureth-2 | | | | |
| Sodium Chloride | — | 1.5 | — | 1.5 |

(7-8) shower bath, (9) shower gel, (10) wash lotion

Cosmetic preparations “2-in-1” shower bath (water, preservative to 100% by weight)

| Composition (INCI) | 11 | 12 | 13 | 14 |
|---|---|---|---|---|
| Texapon ® NSO | 30.0 | 25.0 | | 25.0 |
| Sodium Laureth Sulfate | | | | |
| Plantacare ® 818 | | | | 8.0 |
| Coco Glucosides | | | | |
| Plantacare ® 2000 | | 8.0 | | |
| Decyl Glucoside | | | | |
| Plantacare ® PS 10 | | | 20.0 | |
| Sodium Laureth Sulfate (and) Coco Glucosides | | | | |
| Dehyton ® PK 45 | | 10.0 | 10.0 | |
| Cocamidopropyl Betaine | | | | |
| Lamesoft ® PO 65 | 5.0 | | | |
| Coco-Glucoside (and) Glyceryl Oleate | | | | |
| Lamesoft ® LMG | | 5.0 | 5.0 | |
| Glyceryl Laurate (and) Potassium Cocoyl Hydrolyzed Collagen | | | | |
| Gluadin ® WQ | 3.0 | | | |
| Laurdimonium Hydroxypropyl Hydrolyzed Wheat Protein | | | | |
| Gluadin ® WK | | | | |
| Sodium Cocoyl Hydrolyzed Wheat Protein | | | | |
| Euperlan ® PK 3000 AM | 5.0 | 3.0 | 4.0 | — |
| Glycol Distearate (and) Laureth-4 (and) Cocamidopropyl Betaine | | | | |
| Panthenol | 0.5 | — | — | 0.5 |
| Pistia stratiotes extract | 1.0 | 1.0 | 1.0 | 1.0 |
| Arlypon ® F | 2.6 | 1.6 | — | 1.0 |
| Laureth-2 | | | | |
| Sodium Chloride | — | — | — | — |

Cosmetic preparations shampoo (all quantities in % by weight, based on the
cosmetic preparation, water, preservative add up to 100% by weight)

| Composition (INCI) | 15 | 16 | 17 | 18 | 19 | 20 |
|---|---|---|---|---|---|---|
| Texapon ® NSO | 30.0 | | | 30.0 | 25.0 | |
| Sodium Laureth Sulfate | | | | | | |
| Texapon ® K 14 S | | 30.0 | | | | 30.0 |
| Sodium Myreth Sulfate | | | | | | |
| Texapon ® SB 3 | | 10.0 | | | | |
| Disodium Laureth Sulfosuccinate | | | | | | |
| Pantacare ® 818 | 4.0 | | | | | |
| Coco Glucosides | | | | | | |
| Plantacare ® 2000 | | 4.0 | | | | |
| Decyl Glucoside | | | | | | |
| Pantacare ® PS 10 | | | 20.0 | | | |
| Sodium Laureth Sulfate (and) Coco Glucosides | | | | | | |
| Dehyton ® PK 45 | 5.0 | | | 10.0 | | 10.0 |
| Cocamidopropyl Betaine | | | | | | |
| Gluadin ® WK | | | | | 8.0 | |
| Sodium Cocyl Hydrolyzed Wheat Protein | | | | | | |
| Lamesoft ® PO 65 | — | — | — | — | 2.0 | 2.0 |
| Coco-Glucoside (and) Glyceryl Oleate | | | | | | |
| Nutrilan ® Keratin W | 5.0 | — | — | — | | — |
| Hydrlozyed Keratin | | | | | | |
| Gluadin ® W 40 | — | 2.0 | — | 2.0 | — | — |
| Hydrolyzed Wheat Protein | | | | | | |
| Euperlan ® PK 3000 AM | — | — | — | 3.0 | 3.0 | — |
| Glycol Distearate (and) Laureth-4 (and) Cocamidopropyl Betaine | | | | | | |
| Panthenol | — | ‘3 | — | — | — | 0.2 |
| Pistia stratiotes extracts | 1.0 | 1.0 | 1.0 | 1.0 | 1.0 | 1.0 |
| Arlypon ® F | 1.5 | — | — | — | — | — |
| Laureth-2 | | | | | | |
| Sodium Chloride | — | 1.6 | 2.0 | 2.2 | — | 3.0 |

Cosmetic preparations foam bath (all quantities in % by weight, based on the
cosmetic preparation, water, preservative add up to 100% by weight)

| Composition (INCI) | 21 | 22 | 23 | 24 | 25 |
|---|---|---|---|---|---|
| Texapon ® NSO | — | 30.0 | 30.0 | — | 25.0 |
| Sodium Laureth Sulfate | | | | | |
| Plantacare ® 818 | — | 10.0 | — | — | 20.0 |
| Coco Glucosides | | | | | |
| Plantacare ® PS 10 | 22.0 | — | 5.0 | 22.0 | 20.0 |
| Sodium Laureth Sulfate (and) Coco Glucosides | | | | | |
| Dehyton ® PK 45 | 15.0 | 10.0 | 15.0 | 15.0 | 15.0 |
| Cocamidopropyl Betaine | | | | | |
| Monomuls ® 90-O 18 | 0.5 | | | | |
| Glyceryl Oleate | | | | | |
| Lamesoft ® PO 65 | | 3.0 | | 3.0 | 2.0 |
| Coco-Glucoside (and) Glyceryl Oleate | | | | | |
| Cetiol ® HE | | | 2.0 | | 2.0 |
| PEG-7 Glyceryl Cocoate | | | | | |
| Nutrilan ® I-50 | 5.0 | | | | |
| Hydrolyzed Collagen | | | | | |
| Gluadin ® W 40 | | 5.0 | | 5.0 | |
| Hydrolyzed Wheat Gluten | | | | | |
| Gluadin ® WK | | | | 7.0 | |
| Sodium Cocoyl Hydrolyzed Wheat Protein | | | | | |
| Euperlan ® PK 3000 AM | 5.0 | — | — | 5.0 | — |
| Glycol Distearate (and) Laureth-4 (and) Cocamidopropyl Betaine | | | | | |
| Arlypon ® F | | | 1.0 | | |
| Laureth-2 | | | | | |
| Sodium Chloride | 1.0 | | 1.0 | | 2.0 |
| Pistia stratiotes extracts | 1.0 | 1.0 | 1.0 | 1.0 | 1.0 |

All substances with the registered trade mark symbol ® used and listed in Table 8 are marks and products of the COGNIS Group.

1. Preparations containing an extract of the plant Pistia stratiotes.

2. Preparations as claimed in claim 1, characterized in that they contain the plant extract in quantities of 0.01 to 25% by weight, based on the total quantity of the preparations, with the proviso that the quantities mentioned add up to 100% by weight with water and optionally other auxiliaries and additives.

3. Preparations as claimed in claim 1, characterized in that they contain substances selected from the group consisting of sterols, carotinoids, proteins, carbohydrates, fats, vitamins and mineral salts.

4. The use of extracts of the plant Pistia stratiotes in skin care and/or hair care preparations.

5. The use of extracts of the plant Pistia stratiotes in care preparations with slimming and anti-cellulitis properties for the skin.

6. The use of extracts of the plant Pistia stratiotes in care preparations for the preventive or healing treatment of signs of skin ageing.

7. The use claimed in claim 6, characterized in that UV-induced skin ageing is treated.

8. The use claimed in claim 6, characterized in that apoptosis induced by a deficiency of growth factors and correspondingly induced signs of skin ageing are treated.

9. The use of extracts of the plant Pistia stratiotes in protective and restorative care preparations with revitalizing and reactivating activity for the skin and/or hair

10. A process for the preparation of an extract of the plant Pistia stratiotes, characterized in that solvents or mixtures of solvents selected from the group consisting of distilled or non-distilled water, low molecular weight alcohols, esters, ethers, hydrocarbons, ketones or halogenated hydrocarbons are used as the extraction medium for extraction.
